(19) United States
(12) Patent Application Publication
(10) Pub. No.: US 2025/0114040 A1
(43) Pub. Date: Apr. 10, 2025

(54) WEARABLE DEVICE

(71) Applicant: GOERTEK INC., Weifang, Shandong (CN)

(72) Inventor: Jie YANG, Weifang, Shandong (CN)

(21) Appl. No.: 18/294,580

(22) PCT Filed: Nov. 2, 2021
(86) PCT No.: PCT/CN2021/128016

§ 371 Date: Feb. 2, 2024

(30) Foreign Application Priority Data
Sep. 27, 2021 (CN) 202111139550.8

(51) Int. Cl. A61B 5/00; A44C 5/00; A61B 5/1455; A61B 5/256; A61B 5/282

(52) U.S. Cl. CPC A61B 5/681; A44C 5/0053; A61B 5/14552; A61B 5/256; A61B 5/282

(57) ABSTRACT

A wearable device, including: a device body provided with a controller; two strap bodies, one end of each of the two strap bodies respectively being connected to either end of the device body, and the other end of each of the two strap bodies being connected to each other and enabling the two strap bodies to partially overlap; a tightness adjustment mechanism arranged on the strap bodies, wherein an overlapping length of the two strap bodies is changeable by adjusting the tightness adjustment mechanism so as to adjust the tightness of the wearable device; and a length measurement mechanism electrically connected to the controller and configured for measuring the overlapping length of the two strap bodies. Thus provided is a wearable device capable of more accurately adjusting tightness.

The present disclosure claims the priority to the Chinese Patent Application No. 202111139550.8, entitled “WEARABLE DEVICE” filed with China Patent Office on Sep. 27, 2021, the entire contents of which are incorporated into the present disclosure by reference.

### TECHNICAL FIELD

The present disclosure relates to a technical field of wearable devices, and more particularly, to a wearable device.

### DESCRIPTION OF RELATED ART

With the development of electronic technology, wearable devices are becoming more and more popular among people. Wearable devices generally include a device body and a strap body, the device body is configured to display image information to users, and the strap body is used for users to wear the device body on their heads, hands, or feet. In order to fit different users, a length of the strap needs to be adjusted so as to adjust the tightness of the wearable device. However, in current wearable devices, the accuracy of tightness adjustment is low, and users often need multiple operations to adjust to an appropriate tightness, which greatly affects the user experience.

### SUMMARY

A main purpose of the present disclosure is to provide a wearable device that can adjust tightness more accurately.

In order to achieve the above purpose, the present disclosure provides a wearable device including:
a device body provided with a controller;
two strap bodies, one end of each of the two strap bodies respectively being connected to either end of the device body, and the other end of each of the two strap bodies being connected to each other and enabling the two strap bodies to partially overlap;
a tightness adjustment mechanism provided on the strap bodies, wherein an overlapping length of the two strap bodies is changeable by adjusting the tightness adjustment mechanism so as to adjust the tightness of the wearable device; and
a length measurement mechanism electrically connected to the controller and configured for measuring the overlapping length of the two strap bodies.

Optionally, the strap bodies include a first strap body and a second strap body, and the length measurement mechanism includes:
a resistance element provided on the second strap body and electrically connected to the controller;
a conductive member provided on the first strap body, one end of the conductive member being electrically connected to the controller, and one end thereof being configured to conductively connect the resistance element; and
a resistance detection element electrically connected to the controller,
wherein the conductive member is conductively connected to the resistance element, and a detection circuit is formed among the controller, the resistance detection element, the conductive member and the resistance element.

Optionally, the resistance element is arranged in a long strip shape extending along a length direction of the second strap body. a length of the resistance element connected to the detection circuit is different according to different overlapping lengths of the first strap body and the second strap body.

Optionally, the tightness adjustment mechanism includes an adjustment member provided at an end of the first strap body, and a rack structure provided at the second strap body, the adjustment member is provided with an adjustment cavity, and at least the rack structure enters or exits the adjustment cavity in a movable manner.

The tightness adjustment mechanism further includes a buckle member rotatably provided in the adjustment cavity to have a first state and a second state.

In the first state, the buckle member is engaged with the rack structure and drives the adjustment member to move, thereby driving the buckle member to move relative to the rack structure and change the length of the rack structure extending into or out of the adjustment cavity so as to increase the length of the second strap body overlapping the first strap body.

In the second state, the buckle member is configured to be separated from the rack structure, thereby moving the second strap body in a direction of exiting the adjustment cavity so as to reduce the length of the second strap body overlapping the first strap body.

Optionally, the resistance element at least has a portion arranged corresponding to the rack structure, and the rack structure includes a plurality of tooth slots spaced apart from each other.

The conductive member is the buckle member, and in the first state, the buckle member is engaged in the tooth slots and is configured to be conductively connected to the resistance element, so that the detection circuit is formed among the controller, the resistance element, the buckle member and the resistance detection element.

The buckle member is configured to be engaged in different tooth slots, so that the overlapping length of the two strap bodies is changeable, and the buckle member is configured to be conductively connected to different positions of the resistance element to change a resistance of the resistance element connected to the detection circuit, such that a detection value of the resistance detection element corresponds to the current overlapping length.

Optionally, a first conductive contact electrically connected to the resistance element is provided in the tooth slots, the buckle member is engaged in the tooth slots and is configured to conductively contact the first conductive contact.

Optionally, a surface of the resistance element is exposed at a bottom of the tooth slots, and the buckle member is engaged in the tooth slots and is configured to conductively contact the resistance element.

Optionally, when the resistance element is in a long strip shape, the resistance element is made of a memory metal material, and the resistance element has an arc-shaped memory state in a natural state, so that the second strap body is at least partially naturally curved.

Optionally, the tightness adjustment mechanism further includes a rotating shaft rotatably provided on the adjustment member, and the buckle member is fixedly connected to the rotating shaft.

Optionally, a mounting hole is provided on each of opposite sides of the adjustment member, and opposite ends of the rotating shaft are installed in the respective mounting holes. At least one end of the rotating shaft is provided with a toggle portion, the toggle portion is exposed outside the adjustment member, and the toggle portion is configured to be driven to allow the buckle member to switch between the first state and the second state.

Optionally, the tightness adjustment mechanism further includes an elastic member, and the elastic member is configured to act on the buckle member, so that the buckle member has a tendency to move towards the first state in the second state.

Optionally, the elastic member is a torsion spring sleeved on the rotating shaft, the torsion spring includes a spring body and a first torsion arm and a second torsion arm respectively connected to opposite ends of the spring body. The first torsion arm is fixedly connected to the adjusting member, and the second torsion arm is configured to abut against the buckle member.

Optionally, there are two torsion springs, the two torsion springs are respectively provided on opposite sides of the buckle member, and the second torsion arms of the two torsion springs are integrally connected to each other.

Optionally, the rack structure includes a plurality of tooth slots spaced apart from each other, the buckle member has an engaging end, and in the first state, the engaging end is obliquely engaged in the tooth slots.

The tooth slot has opposite first and second sides, the buckle member moves from the first side of one tooth slot to another adjacent tooth slot to increase the overlapping length of the two strap bodies.

On the first side, each of the tooth slots is provided with a guide slope parallel to the engaging end, and the guide slope is configured to guide the engaging end to enter another adjacent tooth slot from the first side.

Optionally, on the second side, each tooth slot is provided with a limiting surface, and the limiting surface is arranged at an acute or right angle to a bottom wall of the tooth slot to restrict the engaging end from entering another adjacent tooth slot from the second side.

Optionally, the wearable device further includes a tightness sensing member electrically connected to the controller, the tightness sensing member is provided on a wearing side of the wearable device and is configured to detect the tightness of the wearable device.

Optionally, the tightness sensing member is a pressure sensor, and the tightness of the wearable device is represented by a pressure value detected by the pressure sensor.

Optionally, a pressure value detected by the pressure sensor corresponds to an overlapping length of the two strap bodies, the controller is configured to obtain a target overlapping length according to a target pressure value, and obtain the current overlapping length detected by the length measurement mechanism, so as to obtain a length adjustment value according to the target overlapping length and the current overlapping length, so that the tightness adjustment mechanism adjusts the overlapping length of the two strap bodies to the target overlapping length according to the length adjustment value.

Optionally, the pressure sensor is also configured to detect an actual pressure value after the overlapping length of the two strap bodies is adjusted to the target overlapping length. The controller obtains the actual pressure value and determines whether a difference between the actual pressure value and the target pressure value is less than or equal to a preset pressure difference.

Optionally, the target pressure value includes a test pressure value. The wearable device is a wrist-worn device, and when the wrist-worn device is at a tightness corresponding to the test pressure value, the wrist-worn device can be configured for testing blood oxygen saturation.

Optionally, the wearable device further includes a prompter electrically connected to the controller, the prompter includes at least one of a display screen, a speaker, or a vibrator, and the prompter is configured to receive and feedback an adjustment instruction generated by the controller.

Optionally, the wearable device is a wrist-worn device, and the wrist-worn device has an electrocardiogram (ECG) monitoring function.

The tightness adjustment mechanism includes an adjustment member provided at an end of one of the strap bodies, and the other strap body is movably connected to the adjustment member. A monitoring electrode electrically connected to the controller is located outside the device body and/or the adjustment member, and at least two monitoring electrodes are provided for ECG monitoring.

The wrist-worn device has a wearing side, at least one of the monitoring electrodes is arranged on the wearing side, and at least one of the monitoring electrodes is arranged on other positions than the wearing side.

Optionally, the monitoring electrode includes a first monitoring electrode arranged on the wearing side and a second monitoring electrode arranged at another position.

The device body is at least provided with one first monitoring electrode and one second monitoring electrode, and the adjustment member is provided with at least one first monitoring electrode.

Optionally, the device body is provided with at least one first monitoring electrode, and the adjustment member is provided with at least one second monitoring electrode.

Optionally, the two strap bodies are detachably connected to the device body, one end of the strap body is provided with a buckling protrusion, and opposite ends of the device body are each provided with a buckling groove, and the buckling groove is engaged with the buckling protrusion.

The buckling protrusion provided on at least one of the strap bodies is a conductive buckling protrusion, electronic components on the strap body are electrically connected to the conductive buckling the protrusion, buckling groove corresponding to the conductive buckling protrusion is provided with a second conductive contact therein, the second conductive contact is electrically connected to the controller, and the conductive buckling protrusion is configured conductively contact the second conductive contact after being engaged with the buckling groove.

Optionally, the second conductive contact is provided on a conductive ejector pin or a conductive elastic piece in the buckling groove, one end of the conductive ejector pin or the conductive elastic piece is connected to a wall of the buckling groove and is electrically connected to the controller, and the other end thereof is provided with the second conductive contact for conductively contacting the conductive buckling protrusion.

In the technical solution of the present disclosure, the size of a wearing ring can be changed by changing the overlapping length of the two strap bodies, and therefore, when a user wears the wearable device after determining the wearing ring with the highest comfortability, he or she can first make a rough adjustment and the length measurement mechanism can detect the current overlapping length of the two strap bodies, and a difference between the current overlapping length and the optimal overlapping length is a length adjustment value. Since the length measurement mechanism is electrically connected to the controller, the controller obtains the current overlapping length and an optimal overlapping length, thereby obtaining a length adjustment value and feeding it back to the user. The user can change the overlapping length of the two strap bodies by adjusting the tightness adjustment mechanism, the adjustment amount corresponds to the length adjustment value, and the overlapping length of the two strap bodies can be adjusted to the optimal overlapping length. As a result, the wearing ring can also be adjusted to the size of the highest comfortability. In this way, the wearable device of the present disclosure can adjust the tightness more accurately, thereby improving the user's wearing comfortability.

### BRIEF DESCRIPTION OF DRAWINGS

In order to more clearly illustrate the embodiments of the present disclosure or the technical solutions in the prior art, the drawings required to be used for the content of the embodiments or the prior art will be briefly introduced in the following. Obviously, the drawings in the following description are merely some embodiments of the present disclosure, and for those of ordinary skill in the art, other drawings can also be obtained from the provided drawings without any creative effort.

FIG. 1 is a structural schematic diagram of a wearable device from a perspective according to an embodiment of the present disclosure;

FIG. 2 is a structural schematic diagram of the wearable device from another perspective according to an embodiment of the present disclosure;

FIG. 3 is an exploded view of the wearable device according to an embodiment of the present disclosure;

FIG. 4 is an exploded view of the wearable device according to another embodiment of the present disclosure;

FIG. 5 is an exploded view of the wearable device according to still another embodiment of the present disclosure;

FIG. 6 is a cross-sectional view of a tightness adjustment mechanism according to an embodiment of the present disclosure;

FIG. 7 is a partial structural schematic diagram of the tightness adjustment mechanism according to an embodiment of the present disclosure;

FIG. 8 is another partial structural schematic diagram of the tightness adjustment mechanism according to an embodiment of the present disclosure;

FIG. 9 is an exploded view of the tightness adjustment mechanism according to an embodiment of the present disclosure;

FIG. 10 is a partial cross-sectional view of the wearable device according to an embodiment of the present disclosure;

FIG. 11 is a partial cross-sectional view of the wearable device according to another embodiment of the present disclosure.

### EXPLANATION OF REFERENCE NUMERALS

| Numeral | Name |
|---|---|
| 100 | device body |
| 110 | resistance |
| | detection |
| | element |
| 120 | Hall element |
| 101 | first end |
| 102 | second end |
| 103 | buckling groove |
| 300 | second strap |
| | body |
| 310 | rack structure |
| 311 | tooth slot |
| 312 | guide slope |
| 313 | limiting |
| | surface |
| 320 | resistance |
| | element |
| 301 | second strap |
| | main body |
| 302 | memory metal |
| | portion |
| 410 | first |
| | monitoring |
| | electrode |
| 420 | second |
| | monitoring |
| | electrode |
| 510 | conductive |
| | elastic piece |
| 520 | conductive |
| | ejector pin |
| p | wearing side |
| 200 | first strap |
| | body |
| 201 | buckling |
| | protrusion |
| 210 | adjustment |
| | member |
| 211 | adjustment |
| | cavity |
| 212 | mounting hole |
| 220 | buckling member |
| 221 | engaging end |
| 230 | rotating shaft |
| 231 | toggle portion |
| 240 | torsion spring |
| 241 | first torsion |
| | arm |
| 242 | second torsion |
| | arm |
| 243 | spring body |
| 250 | color sensor |
| 260 | pressure sensor |
| 270 | magnet |
| a | first side |
| b | second side |

The realization of the purpose, functional features and advantages of the present disclosure will be further described with reference to the embodiments in combination with the accompanying drawings.

### DETAILED DESCRIPTIONS

Technical solutions of embodiments of the present disclosure will be clearly and completely described below with reference to the drawings in the embodiments of the present disclosure. Obviously, the described embodiments are only a part of the embodiments of the present disclosure, rather than all the embodiments. Based on the embodiments in the present disclosure, all other embodiments obtained by those of ordinary skill in the art without creative efforts shall fall within the protection scope of the present disclosure.

It should be noted that all directional indications (such as up, down, left, right, front, back . . . ) in the embodiments of the present disclosure are only configured to explain the relative positional relationships and movement conditions, etc. among the components in a specific posture (as shown in the accompanying drawings), and if the specific posture changes, the directional indication will also be changed accordingly.

In addition, “first”, “second”, etc. in the present disclosure are only for descriptive purposes, and should not be construed as indicating or implying their relative importance or implicitly indicating the quantity of indicated technical features. Thus, the features defined with “first”, “second”, etc. may explicitly or implicitly include at least one of these features. In addition, the meaning of “and/or” herein includes three parallel schemes. For example, the meaning of “A and/or B” includes a scheme A, a scheme B, or a scheme of both A and B. In addition, the technical solutions of various embodiments of the present disclosure can be combined with each other, but it should be based on the fact that the technical solutions can be realized by those skilled in the art. When the combination of technical solutions is contradictory or cannot be realized, it should be considered that the combination does not exist and is not within the scope of protection required by the present disclosure.

The present disclosure provides a wearable device.

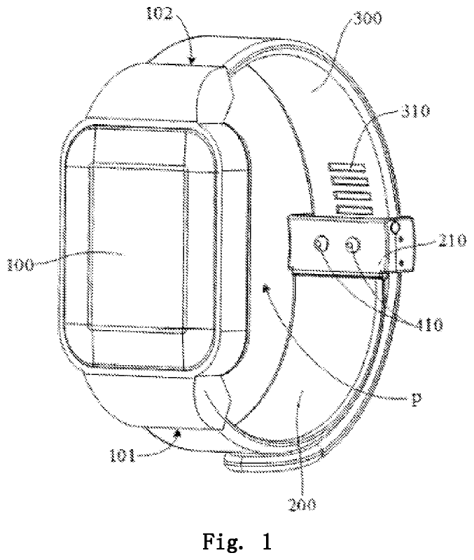

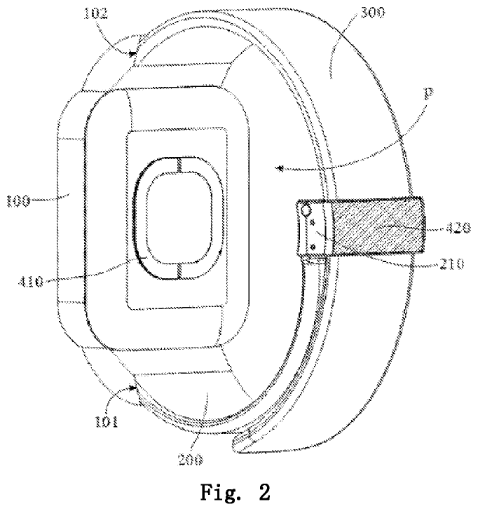

In an embodiment of the present disclosure, as illustrated in FIGS. 1 and 2, the wearable device includes:
a device body 100 provided with a controller;
two strap bodies, including a first strap body 200 and a second strap body 300, one end of each of the two strap bodies respectively being connected to either end of the device body 100, and the other end of each of the two strap bodies being connected to each other and enabling the two strap bodies to partially overlap;
a tightness adjustment mechanism provided on the strap bodies, wherein an overlapping length of the two strap bodies is changeable by adjusting the tightness adjustment mechanism so as to adjust the tightness of the wearable device; and
a length measurement mechanism electrically connected to the controller and configured for measuring the overlapping length of the two strap bodies.

It will be understood that the two ends of the strap body are a free end and a fixed end, the fixed end of the strap body is configured to connect to an end of the device body 100, and the free ends of the two strap bodies are connected to each other, so that the two strap bodies and the device body 100 are looped together to form a wearing ring. The size of the wearing ring should be adapted to the user's head, hands or feet, so that the tightness of the wearable device meets the user's needs, thereby improving the comfortability of users wearing the wearable device. Here, the wearable device includes watches, smart watches or smart bracelets that can be worn on the wrist, motion monitoring modules that can be worn on the feet, and 3D display devices that can be worn on the head.

In the present disclosure, the size of the wearing ring can be determined based on the overlapping length detected by the length measurement mechanism. Accordingly, an optimal overlapping length is determined, and the wearing ring may also correspond to the size of the highest comfortability. In general, the wearable device of the present disclosure can calibrate the optimal overlapping length in the following two ways:

The first way is to determine the most comfortable wearing ring for the user based on a circumference of a body part where the user wears the wearable device. The second way is to determine the most comfortable wearing ring through the user's own perception when wearing it for the first time. At this time, the overlapping length of the two strap bodies, which is the optimal overlapping length, is detected by the length measurement mechanism.

The size of the wearing ring can be changed by changing the overlapping length of the two strap bodies, and therefore, when the user wears the wearable device after determining the wearing ring with the highest comfort, he or she can first make a rough adjustment and the length measurement mechanism can detect the current overlapping length of the two strap bodies, and a difference between the current overlapping length and the optimal overlapping length is a length adjustment value. Since the length measurement mechanism is electrically connected to the controller, the controller obtains the current overlapping length and an optimal overlapping length, thereby obtaining a length adjustment value and feeding it back to the user. The user can change the overlapping length of the two strap bodies by adjusting the tightness adjustment mechanism, the adjustment amount corresponds to the length adjustment value, and the overlapping length of the two strap bodies can be adjusted to the optimal overlapping length. As a result, the wearing ring can also be adjusted to the size of the highest comfort. In this way, the wearable device of the present disclosure can adjust the tightness more accurately, thereby improving the user's wearing comfort.

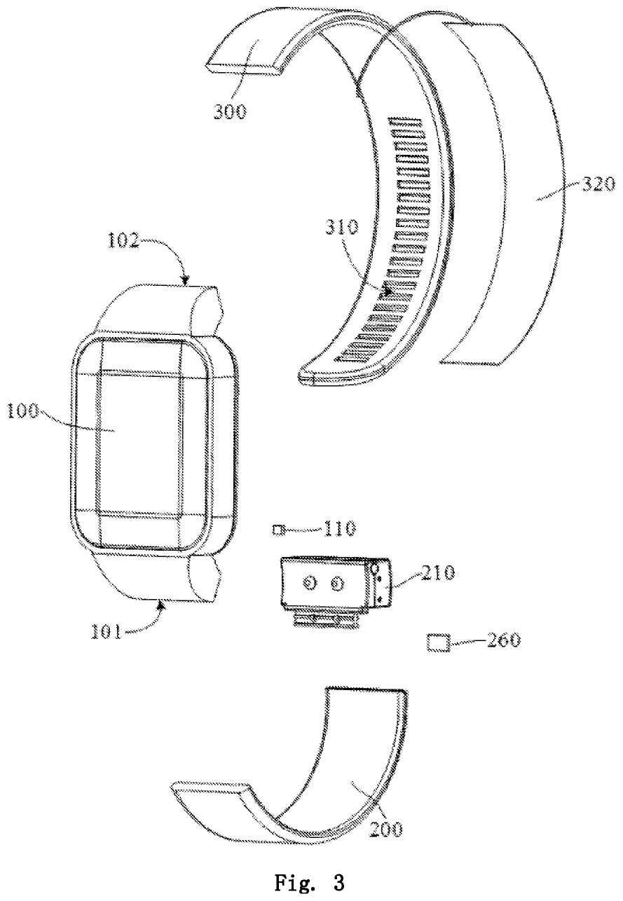

Referring to FIG. 3, in some embodiments, the length measurement mechanism includes:
a resistance element 320 provided on the second strap body 300 and electrically connected to the controller;
a conductive member provided on the first strap body 200, one end of the conductive member being electrically connected to the controller, and one end thereof being configured to conductively connect the resistance element 320; and
a resistance detection element 110 electrically connected to the controller,
wherein the conductive member is conductively connected to the resistance element 320, and a detection circuit is formed among the controller, the resistance detection element 110, the conductive member and the resistance element 320,
wherein the conductive member is conductively connected to different positions of the resistance element 320 according to different overlapping lengths of the two strap bodies, so that the resistance of the resistance element 320 connected to the detection circuit is different, and
wherein the resistance detection element 110 detects the resistance of the resistance element 320 connected to the detection circuit to represent the overlapping length of the two strap bodies.

As such, a resistance value of the resistance detection element 110 may correspond to an overlapping length of the two strap bodies. In the embodiment, the overlapping length is represented by the resistance value, which is convenient, fast and accurate, and can provide precise basis for adjusting the tightness so as to ensure the user's wearing comfortability.

Furthermore, the resistance element 320 is arranged in a long strip shape extending along a length direction of the second strap body 300. a length of the resistance element 320 connected to the detection circuit is different according to different overlapping lengths of the first strap body 200 and the second strap body 300.

It will be understood that each strap body should have a portion for overlapping with another strap body at an end away from the device body 100, the second strap body 300 of at least such portion should be distributed with the resistance element 320 in a long strip shape, and the conductive member should also be disposed on the first strap body 200 of such portion, and within an adjustable range of the overlapping length, the conductive member should always be disposed relative to the resistance element 320 to ensure that the conductive member is configured to be conductively connected to the resistance element 320. As such, when changing the overlapping length of the two strap bodies, the resistance detection element 110 can always detect the resistance value corresponding to the current overlapping length. In particular, when the resistance element 320 is arranged in a uniform long strip structure, the resistance value of the resistance element 320 connected to the detection circuit may be linearly related to the length thereof connected to the detection circuit, making it easier to obtain the length adjustment value.

Of course, the resistance element may also be formed by connecting a plurality of resistance elements in series. the number of resistance elements connected to the detection circuit is different according to different overlapping lengths of the two strap bodies. In this way, the overlapping length of the two strap bodies can also be represented by the resistance value detected by the resistance elements.

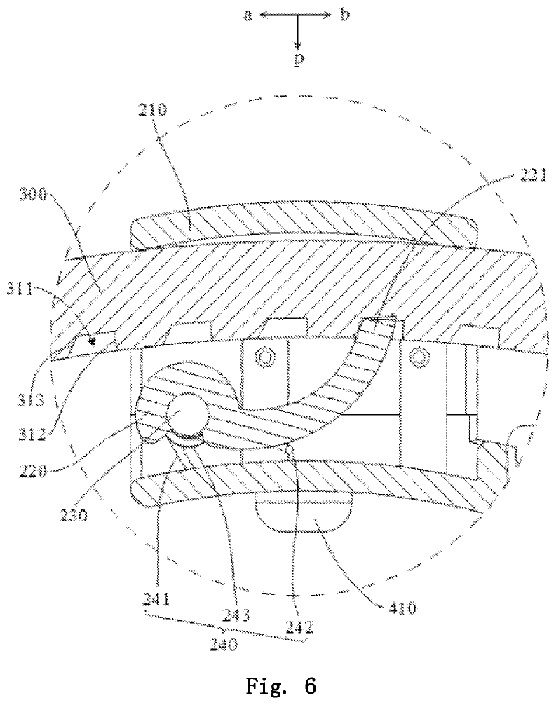

Referring to FIG. 6 to FIG. 9 together, the tightness adjustment mechanism further includes an adjustment member 210 provided at an end of the first strap body 200, and a rack structure 310 provided at the second strap body 300, the adjustment member 210 is provided with an adjustment cavity 211, and at least the rack structure 310 enters or exits the adjustment cavity 211 in a movable manner. The tightness adjustment mechanism further includes a buckle member 220 rotatably provided in the adjustment cavity 211 to have a first state and a second state. In the first state, the buckle member 220 is engaged with the rack structure 310 and drives the adjustment member 210 to move, thereby driving the buckle member 220 to move relative to the rack structure 310 and change the length of the rack structure 310 extending into or out of the adjustment cavity 211, so as to increase the length of the second strap body 300 overlapping the first strap body 200. In the second state, the buckle member 220 is configured to be separated from the rack structure 310, moving the second strap body 300 in a direction of exiting the adjustment cavity 211, so as to reduce the length of the second strap body 300 overlapping the first strap body 200.

In the embodiment, the rack structure 310 includes a plurality of tooth slots 311 spaced apart from each other. After the second strap body 300 is inserted into the adjustment cavity 211, the buckle member 220 is configured to be adjusted to the first state to engage the buckle member 220 in one of the tooth slots 311 of the rack structure 310, and the second strap body 300 is configured to be stably connected to the first strap body 200 without external force.

When it is necessary to increase the overlapping length of the two strap bodies, the user should apply force on the second strap body 300 while applying force to drive the adjustment member 210, so that the buckle member 220 may slide between the tooth slots 311 of the rack structure 310, and that the length of the second strap body 300 extending into or out of the adjustment cavity 211 becomes longer. It will be understood that before the second strap body 300 is fully extended into the adjustment cavity 211, the longer the length of the second strap body 300 extending into the adjustment cavity 211 is, the longer the overlapping length of the two strap bodies is; and after the second strap body 300 is fully extended into the adjustment cavity 211, the longer the length of the second strap body 300 extending out of the adjustment cavity 211 is, the longer the overlapping length of the two strap bodies is.

When it is necessary to reduce the overlapping length of the two strap bodies, the buckle member 220 should be adjusted to the second state, so that the buckle member 220 is separated from the tooth slots 311 of the rack structure 310, and the second strap body 300 can be easily moved towards a direction of exiting the adjustment cavity 211, so as to meet the user's adjustment needs.

Without loss of generality, in the embodiment, the adjustment cavity 211 is provided on the opposite side to a wearing side p of the first strap body 200, so that after the second strap body 300 is inserted into the adjustment cavity 211, the second strap body 300 is configured to be surrounded at an outer side of the first strap body 200, which is convenient for the user to apply force and can improve the user's convenience in adjusting the tightness. The adjustment member 210 is composed of two parts fixedly connected by pins, and the two parts jointly limit the adjustment cavity 211. As such, other components of the tightness adjustment mechanism may be installed in one part of the adjusting member 210, and then the other part of the adjusting member 210 may be fixed, which can improve the assembly convenience of the tightness adjusting mechanism. At this time, the adjustment cavity 211 has no other openings except an inlet and an outlet. Of course, in other embodiments, the adjustment member may also be an integral structure, and may be opened on the side to facilitate the assembly of other components, and the adjusting cavity may have other openings besides the inlet and outlet, as long as the second strap body cannot detach from the adjustment cavity through this opening.

Furthermore, the resistance element 320 at least has a portion corresponding to the rack structure 310; the conductive member is the buckle member 220, the buckle member 220 is engaged in the tooth slots 311 and is configured to be conductively connected to the resistance element 320, so that the detection circuit is formed among the controller, the resistance element 320, the buckle member 220 and the resistance detection element 110; the buckle member 220 is configured to be engaged in different tooth slots 311, so that the overlapping length of the two strap bodies is changeable, and the buckle member 220 is configured to be conductively connected to different positions of the resistance element 320 to change a resistance of the resistance element 320 connected to the detection circuit, such that a detection value of the resistance detection element 110 corresponds to the current overlapping length.

In this way, the cooperation of the buckle member 220 and the tooth slots 311 can not only realize the tightness adjustment of the wearable device, but also connect the resistance element 320 to the detection circuit to ensure that the detection results of the length measurement mechanism can be synchronized with the tightness adjustment mechanism, and to ensure the accuracy of tightness adjustment. In addition, in the present disclosure, an adjustment range of the overlapping length of the two strap bodies is determined by the tooth slots 311 at opposite ends of the rack structure 310 respectively meshing with the buckle member 220. In the embodiment, the buckle member 220 is made of a conductive material, so that the resistance element 320 is conductively connected to the resistance element 320 to ensure that the length measurement mechanism can detect the overlapping length of the two strap bodies within the adjustment range.

Optionally, a first conductive contact electrically connected to the resistance element 320 is provided in the tooth slots 311, the buckle member 220 is engaged in the tooth slots 311 and is configured to conductively contact the first conductive contact. As such, the buckle member 220 is configured to be indirectly conductively connected to the resistance element 320 by contacting the first conductive contact. Optionally, a surface of the resistance element 320 is exposed at a bottom of the tooth slots 311, and the buckle member 220 is engaged in the tooth slots 311 and is configured to conductively contact the resistance element 320. As such, the buckle member 220 is configured to be directly conductively connected to the resistance element 320 by contacting the surface of the resistance element 320.

Furthermore, when the resistance element 320 is in a long strip shape, the resistance element 320 is made of a memory metal material, and the resistance element 320 has an arc-shaped memory state in a natural state, so that the second strap body 300 is at least partially naturally curved. It will be understood that memory metal has super elasticity, which means that memory metal has a much greater deformation recovery ability than ordinary metals under the action of external force, that is, the large strain generated during the loading process may be eliminated when unloading, thereby returning to the memory shape. In the embodiment, the resistance element 320 is made of a memory metal material which can have both electrical conductivity and deformation recovery capabilities, thereby avoiding the deformation of the resistance element 320 which may causes a change of its relative position to the tooth slots 311 and thus the buckle member 220 cannot be conductively connected to the resistance element 320,. Therefore, stability of the length measurement mechanism is ensured.

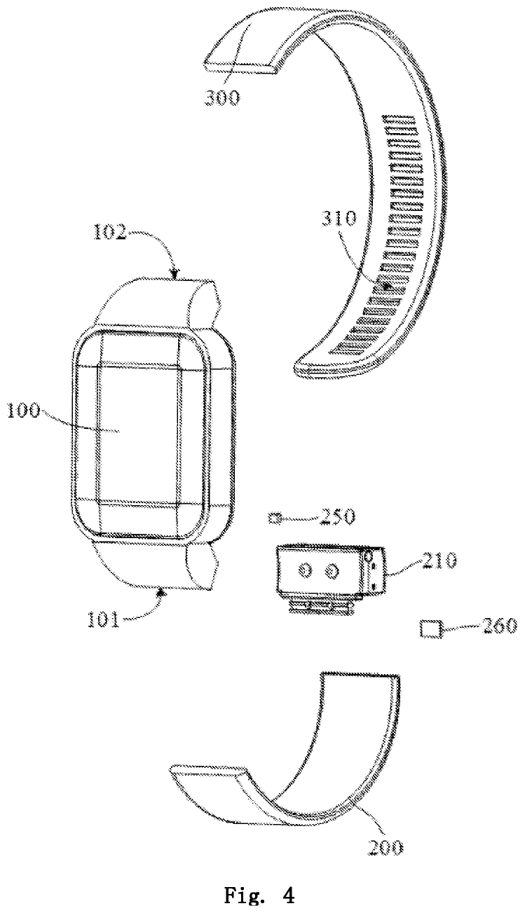

Referring to FIG. 4, in some embodiments, the length measurement t mechanism includes a color sensor 250 and plurality of detection parts respectively provided on the two strap bodies. The color sensor 250 is electrically connected to the controller, and the color of the detection part identified by the color sensor 250 represents the overlapping length of the two strap bodies.

Specifically, the color sensor 250 is provided on the first strap body 200 and the detection part is provided on the second strap body 300. Different detection parts can reflect or emit light of different colors for the color sensor 250 to identify. different detection parts are arranged opposite to the color sensor 250 according to different overlapping lengths of the two strap bodies, so that the color identified by the color sensor 250 corresponds to the overlapping length of the two strap bodies to represent the overlapping length of the two strap bodies using the color identified by the color sensor 250.

The color sensor 250 is a small digital sensor that can convert red, green, and blue components of light into a pulse signal of a certain frequency and can directly measure the RGB color information of an object to be detected, without an A/D converter and an impedance amplifier. The color sensor 250 has high reliability in detecting similar colors and hues, and is widely used in colorimetric analysis.

It will be understood that the detection part should be disposed close to the free end of the second strap body 300, and the color sensor 250 should also be disposed close to the free end of the first strap body 200, so that after the two strap bodies are connected, the color sensor 250 is configured to be disposed opposite the detection part. In the length direction of the second strap body 300, different colors are distributed at different positions of the detection part. As the overlapping length of the two strap bodies changes, the color sensor 250 is positioned at different positions relative to the detection part, and thus different colors is configured to be identified, so that a color identified by the color sensor 250 corresponds to an overlapping length of the two strap bodies, and thus the overlapping length of the two strap bodies is configured to be represented by the recognition result of the color sensor 250. As such, it can also provide a basis for adjusting the tightness conveniently, quickly and accurately to ensure the user's wearing comfortability.

Furthermore, a plurality of detection parts are provided, and the plurality of detection parts are distributed at different positions along the length direction of the second strap body 300. Optionally, the detection parts at different positions are coated with UV paint or fluorescent powder of different colors, so that different detection parts can reflect light of different colors for identification by the color sensor 250. Optionally, the detection parts at different positions are provided with light sources of different colors, so that different detection parts can emit light of different colors for identification by the color sensor 250.

Furthermore, the tooth slots 311 are the detection parts. Optionally, different tooth slots 311 are coated with UV paint or fluorescent powders of different colors, so that different tooth slots 311 can reflect light of different colors. Optionally, light sources of different colors are provided in different tooth slots 311, so that different tooth slots 311 can emit light of different colors. The color sensor 250 is disposed on the buckle member 220. The buckle member 220 engages in different tooth slots 311 and can change the overlapping length of the two strap bodies. The color sensor 250 can identify different colors, and the color identified by the color sensor 250 corresponds to the current overlapping length.

It will be understood that when the buckle member 220 engages with different tooth slots 311, it can drive the two strap bodies to move relative to each other, so that the overlapping lengths of the two strap bodies may be different, thereby adjusting the tightness of the wearable device; the buckle member 220 is provided with a color sensor 250, different tooth slots 311 can reflect or emit light of different colors, and the color sensor 250 can detect the tooth slot 311 where the buckle member 220 is located by identifying the color of the light from the tooth slot 311, so that it is possible to obtain the overlapping length of the two strap bodies at this time. As such, the cooperation of the buckle member 220 and the tooth slots 311 can not only realize the tightness adjustment of the wearable device, but also enable the length measurement mechanism to detect the overlapping length of the two strap bodies, so as to ensure that the detection results of the length measurement mechanism can be synchronized with the adjustment process of the tightness adjustment mechanism, to ensure the accuracy of tightness adjustment, and also to ensure that the length measurement mechanism can detect the overlapping length of the two strap bodies within the adjustment range.

Optionally, different tooth slots 311 are coated with UV paint or fluorescent powders of different colors. Optionally, light sources of different colors are provided in different tooth slots 311. In this way, different tooth slots 311 can reflect light of different colors for identification by the color sensor 250.

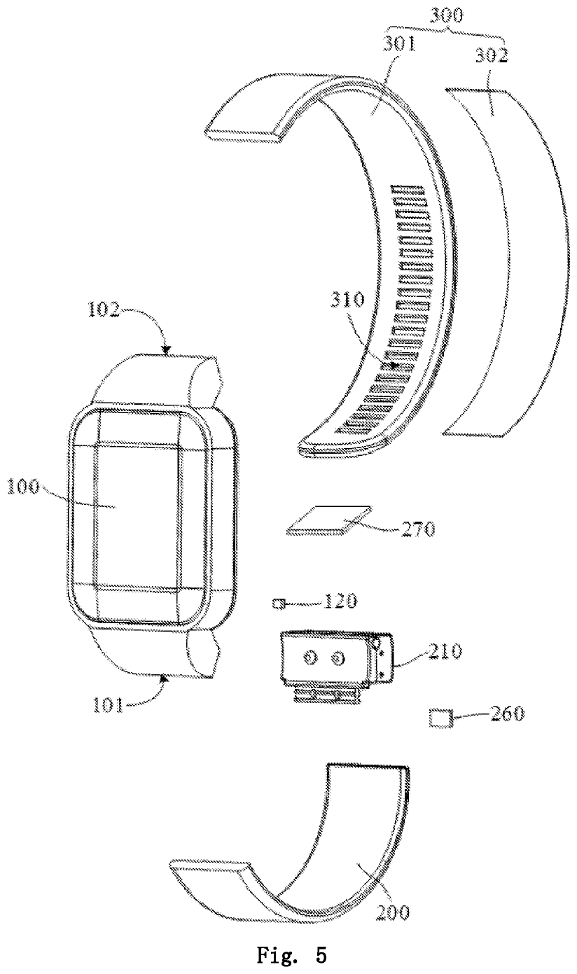

Referring to FIG. 5, in some embodiments, the length measurement mechanism includes a magnet 270 and a Hall element 120 electrically connected to the controller. One of the Hall element 120 and the magnet 270 is disposed on the second strap body 300, and the other one is disposed on one of the device body 100 and the first strap body 200.

Adjusting the tightness adjustment mechanism can change the relative position of the second strap body 300 and the device body 100 and the overlapping length of the two strap bodies so as to change a distance between the magnet 270 and the Hall element 120 and adjust the tightness of the wearable device, so that a Hall voltage generated by the Hall element 120 corresponds to the overlapping length of the two strap bodies, and thus the overlapping length of the two strap bodies is represented by the Hall voltage generated by the Hall element 120.

It will be understood that after the two strap bodies are connected, the overlapping length of the two strap bodies changes, and the tightness of the wearable device can be adjusted. At the same time, the relative position of the two strap bodies may also change. Specifically, free ends of the two strap bodies may change. Moreover, at least the relative position between the second strap body 300 and the device body 100 may also change. Specifically, the distance between the free end of the second strap body 300 and the end of the device body 100 may change.

In the embodiment, one of the Hall element 120 and the magnet 270 is disposed on the second strap body 300, and the other one is disposed on the first strap body 200 or the device body 100, that is, the Hall element 120 and the magnet 270 may be respectively provided on two strap bodies, or one is provided on the device body 100 and the other one is provided on a strap body. When the tightness adjustment mechanism is adjusted to change the overlapping length of the two strap bodies, the relative position of the strap body and the device body 100 changes accordingly, thereby ensuring that the distance between the Hall element 120 and the magnet 270 changes synchronously with the overlapping length of the two strap bodies. In this way, a Hall voltage value generated by the Hall element 120 can correspond to an overlapping length of the two strap bodies, so that the Hall voltage generated by the Hall element 120 can represent the overlapping length of the two strap bodies, which can also provide a basis for adjusting the tightness conveniently, quickly and accurately to ensure the user's wearing comfortability.

Furthermore, the Hall element 120 is provided on the device body 100 and the magnet 270 is provided on the second strap body 300. It will be understood that the Hall element 120 needs to be electrically connected to the controller on the device body 100. Thus, the Hall element 120 is disposed on the device body 100 to ensure a stable connection with the controller.

Furthermore, the device body 100 includes a first end 101 and a second end 102 opposite to each other, the first strap body 200 is connected to the first end 101, the second strap body 300 is connected to the second end 102, Hall element 120 is disposed on the first end 101, and the magnet 270 is disposed on an end of the second strap body 300 away from the second end 102. That is, the fixed end of the second strap body 300 is connected to the second end 102 of the device body 100, and the magnet 270 is provided at the free end of the second strap body 300. Specifically, the magnet 270 is embedded in the second strap body 300 to achieve a stable connection between the magnet 270 and the second strap body 300, and the Hall element 120 is disposed on the first end 101 of the device body 100. Accordingly, the distance between the free end of the second strap body 300 and the first end 101 of the device body 100 changes, and the distance between the magnet 270 and the Hall element 120 may also change simultaneously, so that the Hall element 120 can induce different Hall voltages correspondingly.

Furthermore, the second strap body 300 includes a second strap main body 301 and a memory metal portion 302 provided on the second strap main body 301. At least a portion of the second strap main body 301 for overlapping the first strap body 200 is provided with the memory metal portion 302, which has a memory state of being arc-shaped in a natural state, so that the second strap main body 301 is at least partially naturally arc-shaped. In this way, the free end of the second strap body 300 can be prevented from being interferingly displaced under the action of gravity or external force, and the free end of the second strap body 300 can be ensured to be stably attached to the first strap body 200, which is not only beneficial to the induction of the Hall element 120, but also ensures that the Hall voltage generated by the induction of Hall element 120 accurately corresponds to the overlapping length of the two strap bodies. Of course, a buckle may also be provided on the first strap body to constrain the free end of the second strap body, and the same effect can be achieved.

Furthermore, as illustrated in FIGS. 6 to 9, the tightness adjustment mechanism further includes a rotating shaft 230 rotatably provided on the adjustment member 210, and the buckle member 220 is fixedly connected to the rotating shaft 230. It will be understood that opposite ends of the rotating shaft 230 are rotatably connected to the adjustment member 210, and a middle section of the rotating shaft 230 is located in the adjusting cavity 211 for fixed connection of the buckling member 220. As such, the rotational stability of the buckling member 220 can be improved. Of course, in other embodiments, it is also possible that both sides of the buckle member are respectively provided with a connecting protrusion, and the buckle member is rotatably connected to the adjustment member through the two connecting protrusions.

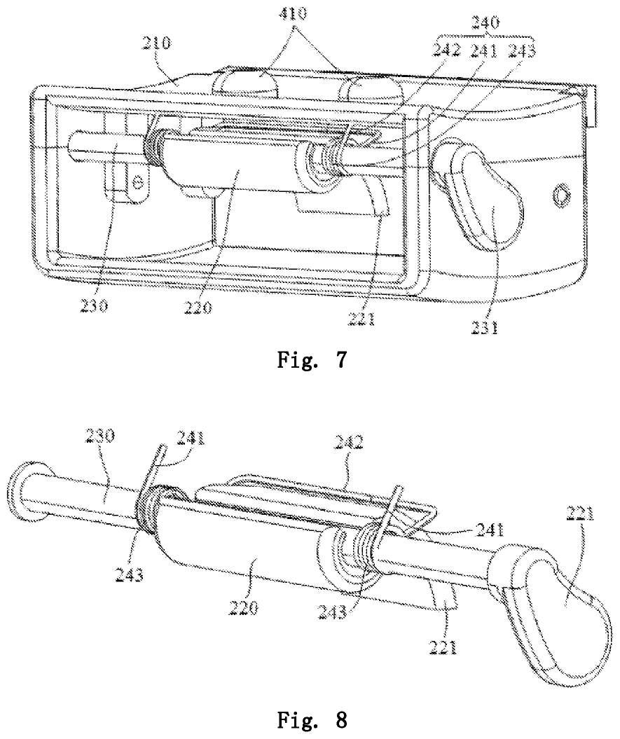

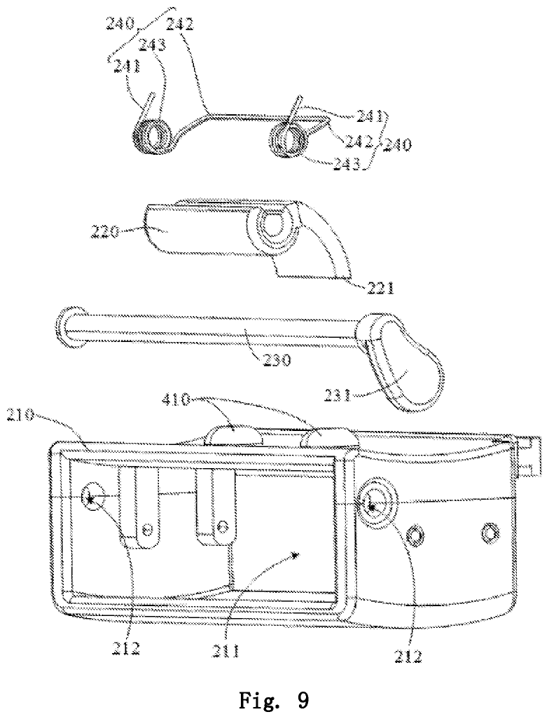

Furthermore, as illustrated in FIG. 9, a mounting hole 212 is provided on each of opposite sides of the adjustment member 210, and opposite ends of the rotating shaft 230 are installed in respective mounting holes 212. As illustrated in FIG. 7, at least one end of the rotating shaft 230 is provided with a toggle portion 231, the toggle portion 231 is exposed outside the adjustment member 210, and the toggle portion 231 is configured to be driven to allow the buckle member 220 to switch between the first state and the second state.

In the embodiment, an end of the rotating shaft 230 is inserted into the mounting hole 212 to penetrate the wall of the adjusting cavity 211, and the toggle portion 231 is provided at one end of the rotating shaft 230, is exposed outside the adjustment member 210 for the convenience of user operation, and is driven by the user to drive the rotating shaft 230 to rotate and finally drive the buckle member 220 to rotate, so that the buckle member 220 can switch between the first state and the second state. Of course, in other embodiments, the adjustment member may be provided with an adjustment hole, and the user inserts a finger or a strip into the adjustment hole to directly act on the buckle member, which can also enable the buckle member to switch between the first state and the second state.

Furthermore, the rack k structure 310 includes a plurality of tooth slots 311 spaced apart from each other, the buckle member 220 has an engaging end 221, and in the first state, the engaging end 221 is obliquely engaged in the tooth slots 311.

The tooth slot 311 has a first side a and a second side b opposite to each other, the buckle member 220 moves from the first side a of one tooth slot 311 to another adjacent tooth slot 311, which can increase the overlapping length of the two strap bodies.

On the first side a, each of the tooth slots 311 is provided with a guide slope 312 parallel to the engaging end 221, and the guide slope 312 is configured to guide the engaging end 221 to enter another adjacent tooth slot 311 from the first side a.

When the user adjusts the tightness adjustment mechanism to increase the overlapping length of the two strap bodies, the user can first fix the adjustment member 210 and then pull the free end of the second strap body 300, so that the tooth slots 311 of the rack structure 310 sequentially slide over the buckle member 220 until it is adjusted in place. When the wearable device is worn on the head or feet, the adjustment member 210 is configured to be fixed with one hand and the second strap body 300 is configured to be pulled with the other hand. When the wearable device is worn on the user's wrist, the adjustment member 210 is configured to be pressed against the desktop first and the second strap body 300 is configured to be pulled with the other hand. Under the guidance of the guide slope 312, the buckle member 220 can smoothly slide through the first side a of the tooth slots 311, and the user can pull the second strap body 300 with less effort, which is beneficial to improving the user's operating experience in adjusting the tightness.

Furthermore, on the second side b, each tooth slot 311 is provided with a limiting surface 313, and the limiting surface 313 is arranged at an acute or right angle to a bottom wall of the tooth slot 311 to restrict the engaging end 221 from entering another adjacent tooth slot 311 from the second side b.

It will be understood that in order to facilitate the user to wear the wearable device, the cooperation between the first strap body 200 and the second strap body 300 is relatively loose in a natural state of the wearable device; after the wearable device is worn on the human body, the cooperation between the first strap body 200 and the second strap body 300 is relatively tight, thus causing the second strap body 300 to have a tendency to separate from the first strap body 200. In the embodiment, the second side b of the tooth slot 311 has a limiting surface 313 arranged at an acute or right angle to the bottom wall of the tooth slot 311 to inhibit the tendency of the second strap body 300 to detach from the first strap body 200 by restricting the engaging end 221 from entering e another adjacent tooth slot 311 from the second side b.

Furthermore, the tightness adjustment mechanism further includes an elastic member that can act on the buckle member 220 so that the buckle member 220 has a tendency to move towards the first state in the second state.

In a natural state of the elastic member, if there is no external force, the buckle member 220 is configured to be stably in the first state, so that the buckle is locked in a tooth slot 311, thereby locking the two strap bodies to ensure that the two strap bodies are in stable connection. Further, when the toggle portion 231 is driven to move the buckle member 220 to the second state, the force exerted on the toggle portion 231 is removed after the overlapping length of the two strap bodies is adjusted. Here, since the elastic member has a tendency to reset to the natural state, it can drive the buckle member 220 to move in a direction of restoring the first state, so that the buckle member 220 can finally be locked in the corresponding tooth slot 311, and the two strap bodies can be locked again. Of course, in other embodiments, a limiting buckle may be provided on the outside of the adjustment member, and when the toggle portion is engaged with the limiting buckle, the buckling member is correspondingly in the first state, and at this time, it is also possible to lock the buckle member.

Furthermore, as illustrated in FIGS. 6 to 9, the elastic member is a torsion spring 240 sleeved on the rotating shaft 230, the torsion spring 240 includes a spring body 243 and a first torsion arm 241 and a second torsion arm 242 respectively connected to opposite ends of the spring body 243. The first torsion arm 241 is fixedly connected to the adjusting member 210, and the second torsion arm 242 is configured to abut against the buckle member 220.

Without loss of generality, in an embodiment of the present disclosure, as illustrated in FIG. 8, when the buckle member 220 is in the first state and engaged in the tooth slot 311 and the torsion spring 240 is in the natural state, there is a certain gap between the buckle member 220 and the second torsion arms 242; when the engaging end 221 slides along the guide slope 312 towards a adjacent tooth slot 311, it is subjected to a reaction force of the guide slope 312, and the buckle member 220 may move towards a direction close to the second torsion arm 242 and can abut against the second torsion arm 242 if the offset is large enough; and after the engaging end 221 enters the tooth slot 311, the shape of the tooth slot 311 and the shape of the engaging end 221 match each other, the engaging end 221 is engaged in the tooth slot 311, and then the buckle member 220 can return to the first state and maintain a certain distance from the second torsion arm 242 of the torsion spring 240 in the natural state. In this way, when the overlapping length of the two strap bodies is increased, the elastic force on the buckle member 220 become smaller, and the user can pull the second strap body 300 with less effort. When the buckle member 220 is in the second state and the torsion spring 240 returns to the natural state, the buckle member 220 cannot be directly driven to the first state; when the torsion spring 240 returns to the natural state, the buckle member 220 will be located in a corresponding position abutting against the second torsion arm 242 until the buckle member 220 is engaged in the tooth slot 311, and then the buckle member 220 may return to the first state. Of course, in other embodiments, it is also possible that in the first state, the buckle member abuts against the second torsion arm.

Furthermore, as illustrated in FIGS. 8 and 9, two torsion springs 240 are provided. The two torsion springs 240 are respectively disposed on opposite sides of the buckle member 220, and the second torsion arms 242 of the two torsion springs 240 are integrally connected to each other. By arranging a torsion spring 240 at opposite ends of the buckle member 220, the force on the buckle member 220 can be more evenly distributed, and connecting the two torsion springs 240 together can prevent the second torsion arm 242 from being deformed due to excessive force between the second torsion arm 242 and the buckle member 220.

Furthermore, the wearable device further includes a tightness sensing member electrically connected to the controller, and the tightness sensing member is provided on the wearing side p of the wearable device for detecting the tightness of the wearable device. The tightness sensing member can detect the tightness of the wearable device, and the tightness of the wearable device is related to the size of the wearing ring and the circumference of the user's wearing part, which can more intuitively reflect the user's wearing comfortability. When the user wears the wearable device for the first time, the controller of the device body 100 may pre-store a preset tightness recommended in ergonomics in order to facilitate the user to determine the optimal size of the wearing ring, and different parts should correspond to different most comfortable preset tightness. When the user adjusts the tightness adjustment mechanism until the detection result of the tightness sensing member corresponds to the preset tightness, the user is allowed to confirm whether it is comfortable, and if the user feels it is loose or tight, he or she can continue to fine-tune, until the optimal tightness considered by the user is reached. As such, the workload of the user in determining the optimal wearing ring can be greatly reduced.

Furthermore, as illustrated in FIGS. 3-5, the tightness sensing member is a pressure sensor 260, and then the tightness of the wearable device is represented by a pressure value detected by the pressure sensor 260. Considering that the device body 100 is not flexible and has poor fit with human skin, the pressure sensor 260 is arranged away from the device body 100 to achieve a better detection effect. For example, the pressure sensor 260 may be disposed on the wearing side p of the first strap body 200, the second strap body 200 or the adjustment member 210. Without loss of generality, in the embodiment, the pressure sensor 260 is disposed on the wearing side p of the first strap body 200 and is disposed close to the adjustment member 210. In this way, after the user wears the wearable device, the pressure sensor 260 may be located between the human skin and the first strap body 200, so that the pressure of the interaction between the two can be sensed. It will be understood that this pressure value is closely related to the tightness of the wearable device, the greater the pressure value is, the tighter the wearable device is worn on the human body. Therefore, the pressure value detected by the pressure sensor 260 can represent the tightness of the wearable device. Of course, in other embodiments, the tightness sensing member may also be a friction sensor, and by slightly rotating the wearable device relative to the wearing part of the human body, the friction sensor detects the friction between the strap body and the human skin so as to represent the tightness of the wearable device.

Furthermore, a pressure value detected by the pressure sensor 260 corresponds to an overlapping length of the two strap bodies, and the controller is configured to obtain a target overlapping length according to a target pressure value, and obtain the current overlapping length detected by the length measurement mechanism so as to obtain a length adjustment value according to the target overlapping length and the current overlapping length, so that the tightness adjustment mechanism adjusts the overlapping length of the two strap bodies to the target overlapping length according to the length adjustment value.

When the user wears the wearable device for the first time, the tightness can be calibrated for different tooth slots 311. First, the buckle member 220 is engaged in a tooth slot 311 closest to the free end of the second strap body 300. At this time, the size of the wearing ring is the largest, and then, by pulling the second strap body 300, the plurality of tooth slots 311 pass through the buckle member 220 in sequence, until the two strap bodies can no longer be tightened or the buckle member 220 reaches a tooth slots 311 farthest from the free end of the second strap body 300. In this process, the length measurement mechanism feeds back the detection values (such as resistance value, color, or Hall voltage, etc.) corresponding to the overlapping length of the two strap bodies to the controller in sequence. At the same time, the pressure sensor 260 feeds back the pressure values to the controller in sequence, and the controller stores the detection value and pressure value corresponding to each tooth slot 311. When the user needs to reach a certain target pressure value, the controller can call a detection value corresponding to the target pressure value to determine a tooth slot 311 that the buckle member 220 needs to reach. At this time, the length measurement mechanism detects the current tooth slot 311 where the buckle member 220 is located, and the controller can give an adjustment instruction after receiving this information. Here, the adjustment instruction includes loosening or tightening, and a specific number of tooth slots 311 to be adjusted. It will be understood that the target pressure value includes the optimal pressure value corresponding to the optimal tightness, and at this time, the corresponding target overlapping length is the optimal overlapping length as well. The user can mark the optimal pressure value, and in daily wearing, the controller may call the overlapping length corresponding to the optimal pressure value and then feed it back to the user. The user can easily adjust the overlapping length of the two strap bodies to the optimal overlapping length by adjusting the tightness adjustment mechanism.

Furthermore, the pressure sensor 260 is also configured to detect an actual pressure value after the overlapping length of the two strap bodies is adjusted to the target overlapping length. The controller obtains the actual pressure value and determines whether a difference between the actual pressure value and the target pressure value is less than or equal to a preset pressure difference. In this way, the actual pressure value detected by the pressure sensor 260 can provide another accurate basis for adjusting the tightness of the wearable device. Before the user adjusts the tightness adjustment mechanism, the current overlapping length detected by the length measurement mechanism is compared with the optimal overlapping length to obtain the length adjustment value, and the length adjustment value can be used as the basis for the user's operation to guide the user to adjust the overlapping length of the two strap bodies. After the user adjusts the tightness adjustment mechanism, the actual pressure value detected by the pressure sensor 260 is compared with the optimal pressure value, and the controller determines whether a difference between the two is within an allowable error range (that is, it does not exceed the preset pressure value), so as to verify whether the tightness of the wearable device is adjusted properly at this time.

Furthermore, the target pressure value includes a test pressure value, and the wearable device is a wrist-worn device. When the wrist-worn device is at a tightness corresponding to the test pressure value, the wrist-worn device can be configured to test blood oxygen saturation.

In the embodiment, the wearable device is a wrist-worn device for wearing on the user's wrist. The wrist-worn device is provided with a blood oxygen detection module for testing the user's blood oxygen saturation. The blood oxygen detection module is mainly based onthe principle that the spectral absorption rates of oxyhemoglobin and deoxyhemoglobin are different according to the Lambert-Beer law, and by using photoplethysmography (PPG) technology, the blood oxygen saturation is derived by measuring the absorption of light of different wavelengths by blood. When testing blood oxygen saturation, the wearable device needs to be worn tightly, but not too tight. It will be understood that when the blood oxygen detection module fits loosely on the wrist, there is a gap between it and the skin of the wrist, or when the fit is too tight and causes compression on the wrist, the light signal cannot reliably reflect signs of blood oxygen saturation, which will lead to inaccurate detection results of the blood oxygen detection module.

In the embodiment, after the calibration is completed, a test pressure value that meets the requirements for testing blood oxygen saturation can be marked. It will be understood that the test pressure value is greater than the optimal pressure value, and, a certain error is allowed in the test of blood oxygen saturation. Thus, the test pressure value should be a range value, as long as it ensures that the measurement result of the blood oxygen saturation is within the allowable error range. When the user needs to test blood oxygen saturation, the controller obtains a tooth slot 311 corresponding to the test pressure value and obtains the current tooth slot 311 fed back by the length measurement mechanism and the current pressure value fed back by the pressure sensor 260 to obtain the number of tooth slots 311 that need to be adjusted. Here, if the current pressure value is greater than the test pressure value, the user is prompted to tighten the corresponding tooth slots 311, and if the current pressure value is less than the test pressure value, the user is prompted to loosen the corresponding tooth slots 311. In this way, the wearable device can be adjusted to a tightness that meets the test requirements for blood oxygen saturation, thereby ensuring the accuracy of detection results of the blood oxygen detection module.

Furthermore, the wearable device further includes a prompter electrically connected to the controller, the prompter includes at least one of a display screen, a speaker, or a vibrator, and the prompter is configured to receive and feedback an adjustment instruction generated by the controller. Optionally, when prompting the number of tooth slots 311 to be adjusted, adjustment information can be displayed on the display screen, or adjustment voice can be broadcast through the speaker; after the tightness is adjusted in place, the user can be prompted to stop adjusting according to vibration of the vibrator. Of course, it is also possible that various prompters work together to provide the user with adjustment information from multiple dimensions, thereby making it more convenient for the user to adjust the tightness.

Furthermore, the wrist-worn device has an ECG monitoring function. As illustrated in FIGS. 1 and 2, the device body 100 and/or the adjustment member 210 are provided with a monitoring electrode electrically connected to the controller on the outside, and at least two monitoring electrodes are provided for ECG monitoring; the wrist-worn device has a wearing side p, at least one of the monitoring electrodes is provided on the wearing side p, and at least one of the monitoring electrodes is provided on other positions than the wearing side p.

That is, the monitoring electrode includes a first monitoring electrode 410 arranged on the wearing side p and a second monitoring electrode 420 arranged at another position. It will be understood that two electrodes, i.e., a wrist electrode and a finger electrode, are needed to realize the ECG monitoring function. The wrist electrode contacts the wrist of the user wearing the wearable device, and the other hand of the user needs to touch the finger electrode. The cooperation of the two electrodes can accurately achieve the ECG monitoring function. In the embodiment, the first monitoring electrode 410 is disposed on the wearing side p of the wrist-worn device, serving as a wrist electrode in contact with the wrist of the user, and the second monitoring electrode 420 is disposed on other positions than the wearing side p, serving as a finger electrode for the user' finger to touch. As such, the accuracy of ECG monitoring can be ensured.

Optionally, the device body 100 is provided with at least one first monitoring electrode 410 and at least one second monitoring electrode 420, and the adjustment member 210 is provided with at least one first monitoring electrode 410. In this way, an ECG signal at the user's wrist can be collected through the first monitoring electrodes 410 at two places, which can further improve the monitoring accuracy of ECG monitoring. In particular, when worn, the wearing side p of the adjustment member 210 can be closer to the skin, with a stronger pressure touch feeling, and it is more stable during measurement and may not cause deformation of the arm due to other hand movements which causes the first monitoring electrode 410 to detach from the skin of the wrist.

Optionally, the device body 100 is provided with at least one first monitoring electrode 410, and the adjustment member 210 is provided with at least one second monitoring electrode 420. In the embodiment, the first monitoring electrode 410 is provided only on the wearing side p of the device body 100, which can free up space on the wearing side p of the wearable device and help further improve the user's wearing comfortability.

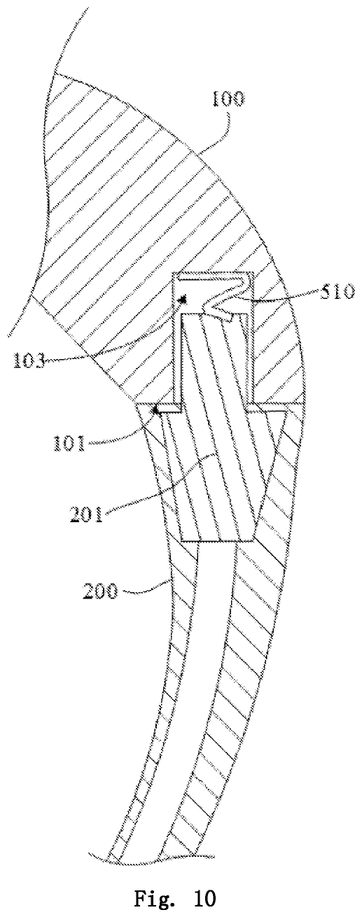

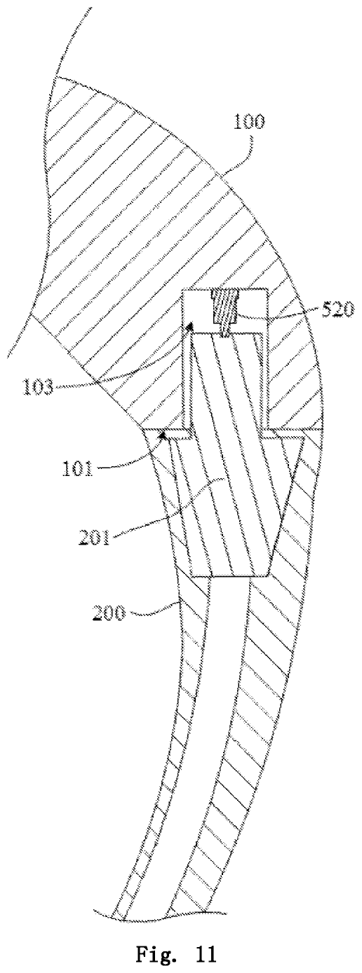

Furthermore, the two strap bodies are detachably connected to the device body 100. As illustrated in FIGS. 9 and 10, one end of the strap body is provided with a buckling protrusion 201, and opposite ends of the device body 100 are each provided with a buckling groove 103, and the buckling groove 103 is engaged with the buckling protrusion 201. The buckling protrusion 201 provided on at least one of the strap bodies is a conductive buckling protrusion 201, electronic components on the strap body are electrically connected to the conductive buckling protrusion 201, the buckling groove 103 corresponding to the conductive buckling protrusion 201 is provided with a second conductive contact therein, the second conductive contact is electrically connected to the controller, and the conductive buckling protrusion 201 is configured to conductively contact the second conductive contact after being engaged with the buckling groove 103.

In the embodiment, the strap body and the device body 100 are connected in a snap-on and detachable manner, so as to improve the maintenance convenience of the wearable device and save the maintenance and replacement cost of the wearable device. Of course, in other embodiments, the strap body and the device body can also be connected through other detachable connection methods, such as plugging, screw locking, etc.

It will be understood that the color sensor 250, the Hall element 120, the pressure sensor 260, the monitoring electrode, etc. can be provided on the strap body. These components provided on the strap body need to be electrically connected to the controller of the device body 100. In the embodiment, the buckling protrusion 201 of the strap body provided with electronic components can be configured as a conductive buckling protrusion 201 that is electrically connected to the electronic components through wires, and the buckle of the device body 100 is provided with a second conductive contact electrically connected to the controller. After the strap body is fixedly connected to the device body 100, the conductive buckling protrusion 201 conductively contacts the second conductive contact, and the electronic components on the strap body can be electrically connected to the controller.

Furthermore, as illustrated in FIGS. 9 and 10, the second conductive contact is provided on a conductive ejector pin 520 or a conductive elastic piece 510 in the buckling groove 103, one end of the conductive ejector pin 520 or the conductive elastic piece 510 is connected to a wall of the buckling groove 103 and is electrically connected to the controller, and the other end thereof is provided with the second conductive contact for conductively contacting the conductive buckling protrusion 201. In this way, when the conductive buckling protrusion 201 is engaged with the buckling groove 103, the conductive buckling protrusion 201 is configured to contact the end of the conductive elastic piece 510 and the conductive ejector pin 520 and be connected with the second conductive contact, so as to realize the electrical connection between the controller and the electronic components on the strap body. Of course, in other embodiments, the second conductive contact may also be disposed on an inner surface of the wall of the buckling groove.

The above are only optional embodiments of the present disclosure and are not intended to limit the patent scope of the present disclosure. Equivalent structural transformations made using the contents of the description and drawings of the present disclosure or direct/indirect applications in other related technical fields are all included in the protection scope of the present disclosure without departing from the inventive concept of the present disclosure.

1. A wearable device comprising:
a device body provided with a controller;
two strap bodies, one end of each of the two strap bodies respectively being connected to either end of the device body, and the other end of each of the two strap bodies being connected to each other and enabling the two strap bodies to partially overlap;
a tightness adjustment mechanism provided on the strap bodies, wherein an overlapping length of the two strap bodies is changeable by adjusting the tightness adjustment mechanism so as to adjust a tightness of the wearable device; and
a length measurement mechanism electrically connected to the controller and configured for measuring the overlapping length of the two strap bodies.

2. The wearable device of claim 1, wherein the strap bodies comprise a first strap body and a second strap body, and wherein the length measurement mechanism comprises:
a resistance element provided on the second strap body and electrically connected to the controller;
a conductive member provided on the first strap body, one end of the conductive member being electrically connected to the controller, and the other end thereof being configured to conductively connect the resistance element; and
a resistance detection element electrically connected to the controller,
wherein the conductive member is conductively connected to the resistance element, and a detection circuit is formed among the controller, the resistance detection element, the conductive member and the resistance element.

3. The wearable device of claim 2, wherein the resistance element is arranged in a long strip shape extending along a length direction of the second strap body and lengths of the resistance element connected to the detection circuit are different according to different overlapping lengths of the first strap body and the second strap body; and/or,
wherein the tightness adjustment mechanism comprises an adjustment member provided at an end of the first strap body, and a rack structure provided at the second strap body, the adjustment member is provided with an adjustment cavity, and at least the rack structure enters or exits the adjustment cavity in a movable manner;
wherein the tightness adjustment mechanism further comprises a buckle member rotatably provided in the adjustment cavity to have a first state and a second state;
wherein in the first state, the buckle member is engaged with the rack structure and drives the adjustment member to move, thereby driving the buckle member to move relative to the rack structure to change a length of the rack structure extending into or out of the adjustment cavity so as to increase the length of the second strap body overlapping the first strap body, and
wherein in the second state, the buckle member is configured to be separated from the rack structure, thereby moving the second strap body in a direction of exiting the adjustment cavity so as to reduce the length of the second strap body overlapping the first strap body.

4. The wearable device of claim 3, wherein the resistance element at least has a portion arranged corresponding to the rack structure, and wherein the rack structure comprises a plurality of tooth slots spaced apart from each other;
wherein the conductive member is the buckle member, and in the first state, the buckle member is engaged in the tooth slots and is configured to be conductively connected to the resistance element, so that the detection circuit is formed among the controller, the resistance element, the buckle member and the resistance detection element; and
wherein the buckle member is configured to be engaged in different tooth slots, so that the overlapping length of the two strap bodies is changeable, and the buckle member is configured to be conductively connected to different positions of the resistance element to change a resistance of the resistance element connected to the detection circuit, such that a detection value of the resistance detection element corresponds to the current overlapping length.

5. The wearable device of claim 4, wherein a first conductive contact electrically connected to the resistance element is provided in each of the tooth slots, and the buckle member is engaged in each of the tooth slots and is configured to conductively contact the first conductive contact; or,
wherein a surface of the resistance element is exposed at a bottom of each of the tooth slots, and the buckle member is engaged in each of the tooth slots and is configured to conductively contact the resistance element;
and/or, wherein when the resistance element is in a long strip shape, the resistance element is made of a memory metal material, and the resistance element has an arc-shaped memory state in a natural state, so that the second strap body is at least partially naturally curved.

6. The wearable device of claim 3, wherein the tightness adjustment mechanism further comprises a rotating shaft rotatably provided on the adjustment member, and the buckle member is fixedly connected to the rotating shaft.

7. The wearable device of claim 6, wherein a mounting hole is provided on each of opposite sides of the adjustment member, and opposite ends of the rotating shaft are installed in respective mounting holes; and wherein at least one end of the rotating shaft is provided with a toggle portion, the toggle portion is exposed outside the adjustment member and is configured to be driven to allow the buckle member to switch between the first state and the second state.

8. The wearable device of claim 6, wherein the tightness adjustment mechanism further comprises an elastic member, and the elastic member is configured to act on the buckle member, so that the buckle member has a tendency to move towards the first state in the second state.

9. The wearable device of claim 8, wherein the elastic member is a torsion spring sleeved on the rotating shaft, and the torsion spring comprises a spring body and a first torsion arm and a second torsion arm respectively connected to opposite ends of the spring body; and wherein the first torsion arm is fixedly connected to the adjusting member, and the second torsion arm is configured to abut against the buckle member.

10. The wearable device of claim 9, wherein two torsion springs are provided, the two torsion springs are respectively provided on opposite sides of the buckle member, and second torsion arms of the two torsion springs are integrally connected to each other.

11. The wearable device of claim 3, wherein the rack structure comprises a plurality of tooth slots spaced apart from each other, the buckle member has an engaging end, and in the first state, the engaging end is obliquely engaged in the tooth slots;
wherein the tooth slot has opposite first and second sides, the buckle member moves from the first side of one tooth slot to another adjacent tooth slot to increase the overlapping length of the two strap bodies;
wherein on the first side, each of the tooth slots is provided with a guide slope parallel to the engaging end, and the guide slope is configured to guide the engaging end to enter another adjacent tooth slot from the first side; and/or,
wherein on the second side, each of tooth slots is provided with a limiting surface, and the limiting surface is arranged at an acute or right angle to a bottom wall of the tooth slot to restrict the engaging end from entering another adjacent tooth slot from the second side.

12. The wearable device of claim 1, wherein the wearable device further comprises a tightness sensing member electrically connected to the controller, and the tightness sensing member is provided on a wearing side of the wearable device and is configured to detect the tightness of the wearable device.

13. The wearable device of claim 12, wherein the tightness sensing member is a pressure sensor, and the tightness of the wearable device is represented by a pressure value detected by the pressure sensor.

14. The wearable device of claim 13, wherein a pressure value detected by the pressure sensor corresponds to an overlapping length of the two strap bodies, and wherein the controller is configured to obtain a target overlapping length according to a target pressure value and obtain the current overlapping length detected by the length measurement mechanism so as to obtain a length adjustment value according to the target overlapping length and the current overlapping length, so that the tightness adjustment mechanism adjusts the overlapping length of the two strap bodies to the target overlapping length according to the length adjustment value.

15. The wearable device of claim 14, wherein the pressure sensor is further configured to detect an actual pressure value after the overlapping length of the two strap bodies is adjusted to the target overlapping length and the controller obtains the actual pressure value and determines whether a difference between the actual pressure value and the target pressure value is less than or equal to a preset pressure difference; and/or,
wherein the target pressure value comprises a test pressure value, the wearable device is a wrist-worn device, and when the wrist-worn device is at a tightness corresponding to the test pressure value, the wrist-worn device is configured for testing blood oxygen saturation.

16. The wearable device of claim 1, wherein the wearable device further comprises a prompter electrically connected to the controller, and the prompter comprises at least one selected from a group consisting of a display screen, a speaker, and a vibrator, and is configured to receive and feedback an adjustment instruction generated by the controller.

17. The wearable device of claim 1, wherein the wearable device is a wrist-worn device, and the wrist-worn device has an electrocardiogram (ECG) monitoring function;
wherein the tightness adjustment mechanism comprises an adjustment member provided at an end of one of the strap bodies, and the other strap body is movably connected to the adjustment member and wherein a monitoring electrode electrically connected to the controller is provided outside the device body and/or the adjustment member, and at least two monitoring electrodes are provided for ECG monitoring; and
wherein the wrist-worn device has a wearing side, at least one of the monitoring electrodes is arranged on the wearing side, and at least one of the monitoring electrodes is arranged on other positions than the wearing side.

18. The wearable device of claim 17, wherein the monitoring electrode comprises a first monitoring electrode arranged on the wearing side and a second monitoring electrode arranged at another position;
wherein the device body is at least provided with one first monitoring electrode and one second monitoring electrode, and the adjustment member is provided with at least one first monitoring electrode; or,
wherein the device body is provided with at least one first monitoring electrode, and the adjustment member is provided with at least one second monitoring electrode.

19. The wearable device of claim 1, wherein the two strap bodies are detachably connected to the device body, one end of each of the strap bodies is provided with a buckling protrusion, and opposite ends of the device body are each provided with a buckling groove, and the buckling groove is engaged with the buckling protrusion; and
wherein the buckling protrusion provided on at least one of the strap bodies is a conductive buckling protrusion, electronic components on the strap body are electrically connected to the conductive buckling protrusion, the buckling groove corresponding to the conductive buckling protrusion is provided therein with a second conductive contact, the second conductive contact is electrically connected to the controller, and the conductive buckling protrusion is configured to conductively contact the second conductive contact after being engaged with the buckling groove.

20. The wearable device of claim 19, wherein the second conductive contact is provided on a conductive ejector pin or a conductive elastic piece in the buckling groove, one end of the conductive ejector pin or the conductive elastic piece is connected to a wall of the buckling groove and is electrically connected to the controller, and the other end thereof is provided with the second conductive contact for conductively contacting the conductive buckling protrusion.
